(19) United States
(12) Patent Application Publication
(10) Pub. No.: US 2025/0090747 A1
(43) Pub. Date: Mar. 20, 2025

(54) Apparatus of Cerebrospinal Fluid Pump

(71) Applicant: Ciatrix, Inc., St Louis Park, MN (US)

(72) Inventor: Zdenko Grajcar, Maple Plain, MN (US)

(21) Appl. No.: 18/966,119

(22) Filed: Dec. 2, 2024

Related U.S. Application Data
(63) Continuation of application No. 18/124,388, filed on Mar. 21, 2023.
(60) Provisional application No. 63/409,619, filed on Sep. 23, 2022.
(60) Provisional application No. 63/354,682, filed on Jun. 22, 2022.
(60) Provisional application No. 63/322,180, filed on Mar. 21, 2022.

(51) Int. Cl. A61M 5/142

(52) U.S. Cl. CPC A61M 5/14276; A61M 2202/0464

(57) ABSTRACT

The present disclosure includes apparatuses and methods for increasing cerebrospinal fluid (CSF) flux. In some embodiments, changes to the curvature of the spinal column results in increased CSF flux. In some embodiments, an implantable pump is used to provide a pressure wave to the CSF. In some embodiments, increased CSF flux resulting in improved clearance of the glymphatic system. In some embodiments, symptoms of Alzheimer's and other neurodegenerative diseases are improved as a result of increased CSF flux.

### CROSS-REFERENCE TO RELATED APPLICATIONS

This application is co-pending with, a continuation of, and claims priority to U.S. patent application Ser. No. 18/124,388, filed Mar. 21, 2023, entitled CEREBROSPINAL FLUID PUMP; and U.S. patent application Ser. No. 18/124,388 claims priority to U.S. Prov. Pat. App. No. 63/322,180, filed Mar. 21, 2022, entitled Cerebrospinal Fluid Pump; U.S. Prov. Pat. App. No. 63/354,682, filed Jun. 22, 2022, entitled Cerebrospinal Fluid Pump; and U.S. Prov. Pat. App. No. 63/409,619, filed Sep. 23, 2022, entitled Cerebrospinal Fluid Pump; and the entire contents of each application are herein incorporated reference.

### TECHNICAL FIELD

This application is directed to devices, systems, and methods for improving cerebrospinal fluid dynamics and for treating diseases associated with dysfunctional cerebrospinal fluid dynamics.

### BACKGROUND

Cerebrospinal fluid (CSF) is a clear, colorless liquid found in the brain and spinal cord, which make up the central nervous system (CNS). The central nervous system controls and coordinates everything that a body can do including muscle movement, organ function, and even complex thinking and planning. CSF acts to cushion the brain and spinal cord when they're struck with mechanical force, to provide basic immunological protection to the CNS, to remove metabolic waste products, including beta amyloid (a protein associated with Alzheimer's disease), as well as to transport nutrients, neuromodulators and neurotransmitters.

Within the brain are the ventricles, which are chambers filled with CSF. There are four ventricles in all: two lateral ventricles, the third ventricle, and the fourth ventricle. CSF is believed to be produced mainly by the choroid plexus epithelium and ependymal cells of the ventricles and flows into interconnecting chambers; namely, the cisterns and the subarachnoid spaces. The ventricles are connected by narrow passageways. CSF flows through the four ventricles and then flows between the meninges in an area called the subarachnoid space. Humans produce approximately 600 ml of CSF every day, continuously replacing it as it is absorbed.

CSF flows from the lateral ventricles through two narrow passageways into the third ventricle. From the third ventricle, it flows down another long passageway known as the aqueduct of Sylvius into the fourth ventricle. From the fourth ventricle, it passes through three small openings called foramina and into the subarachnoid space surrounding the brain and the spinal cord. CSF circulates through a system of cavities found within the brain and spinal cord; ventricles, subarachnoid space of the brain and spinal cord and the central canal of the spinal cord.

It has traditionally been thought that CSF is absorbed through specialized cell clusters called arachnoid villi near the top and midline of the brain. The CSF then passes through the arachnoid villi into the superior sagittal sinus, a large vein, and is absorbed into the bloodstream. Once in the bloodstream, it is carried away and filtered by the kidneys and liver in the same way as other bodily fluids. However, more recent research has shown that CSF is also absorbed through other pathways as well.

The subarachnoid space of the spinal cord is continuous to that of the brain, so that the CSF that is produced in the brain ventricles can easily reach the spinal cord as well. CSF flows from the fourth ventricle into the central canal of the spinal cord through the obex. It passes through the median aperture (of Magendie) and lateral apertures (of Luschka) to enter the interpeduncular and quadrigeminal subarachnoid cisterns. From here, it continues down through the subarachnoid space of the spinal cord. The spinal subarachnoid space is relatively large, accommodating about half of the total volume of CSF in the CNS. It extends from the foramen magnum and ends at the level of the S2 vertebra. Below the conus medullaris, roughly at the level of L1-L2, the subarachnoid space enlarges into a dural sac called the lumbar cistern. The lumbar cistern extends from L1/L2-S2 vertebral levels and it contains the dorsal and ventral rootlets of L2-Co spinal nerves (cauda equina). Given that the spinal and cranial subarachnoid spaces are continuous, the spinal CSF flows back to the cranial subarachnoid space via which it is eliminated into the dural venous sinuses.

CSF is believed to flow due to the forces generated by cardiac pulsations and pulmonary respiration. Some studies have found that CSF flow related to heartbeat was only a minor component of the total flow. Rather respiration is a major regulator of CSF flow. CSF flow rates in response to breathing demonstrate an elevation and lowering of thoracis wall along with positive (toward the brain) and negative (away from the brain) flow corresponding to inspiration and expiration. One study found forced inspiration elicited a prompt and significant increase of CSF flow which was directed upward not only in the cervical and thoracic spine, but also in the aqueduct as part of the brain.

In general flux is defined as the rate of flow of per unit area. As discussed herein, CSF flows both directions depending upon the respiratory stage (inhalation or exhalation) and cardiac stage (systole or diastole). As used herein, flux will mean the effective flow (subtracting the flow in one direction from the flow in the other direction).

The human brain, or the brain parenchyma, is surrounded by interstitial fluid (ISF). Fluid enters the brain parenchyma across the blood/brain barrier and from the CSF system, including from the subarachnoid spaces and ventricles. CSF system is part of the glymphatic system. The fluid entering the brain ISF system contains, in addition to water, also other components, nutrients. Waste products from the brain parenchyma are transferred from the ISF to the CSF system. The waste in the CSF system enters the blood stream through arachnoid villi. It is also believed that there is a path for waste products to flow from the ISF system directly to the lymphatics and also a path for waste products to flow from the CSF system directly to the lymphatics.

Neuroinflammation is an inflammatory response within the brain or spinal cord and occurs when the brain's innate immune system is triggered following an inflammatory challenge such as those posed by injury, infection, exposure to a toxins, aging or other inflammatory agents. Injuries, infections, toxins, and aging are associated with decreased glymphatic system effectiveness. Some studies have shown a rising tau and beta amyloid levels is associated with higher levels of neuroinflammation.

CSF flux is involved in the pathophysiology of neurodegenerative diseases and cognitive impairment. It is known that CSF plays an important role in regulating brain homeostasis, waste clearance, intracranial pressure, and blood and nutrient supply. Whether due to aging, disease, or an accident, CSF flux and overall CSF dynamics can be disrupted which can contribute to the etiology of age-related neurocognitive disorders. These diseases/disorders include Alzheimer's, Parkinson's, Huntington's dementia, prion disease, and multiple sclerosis, brain injury, trigeminal neuralgia, tremors, bipolar disorder, attention deficit disorder, seizures, as well as memory loss, visuo-constructive capacities, and verbal fluency.

As discussed above, two aspects of the cerebrospinal fluid system are the exchange of waste products with the interstitial fluid and the filtering of the CSF to remove these waste products. The CSF/ISF exchange is most active when a person is sleeping or resting. In sleep or during relaxation such meditation, yoga or qigong, a person's blood pressure decreases, and neuronal activity is at minimum, especially in brain frequencies above 8 Hz.

Improved CSF flux and dynamics may improve the health of people with neurocognitive disorders.

A stroke occurs when the blood supply to part of the brain is blocked or when a blood vessel in the brain bursts. In either case, brain cells die, and the brain is damaged temporarily or permanently. Depending on the area of brain deprived of oxygen, a person may experience loss of memory, movement, or speech, or other disabilities. If blood flow is restored or pressure is relieved quickly through medical treatment, the brain may fully recover. A stroke can cause lasting brain damage, long-term disability, or even death.

### SUMMARY OF THE DISCLOSURE

Described herein are apparatus and methods for increasing CSF flux and flow and improving CSF dynamics. In some embodiments, and external, mechanical device is used. In some embodiments, an implantable pump is used.

In one embodiment spinal column flexure is used to increase CSF flux. In some embodiments, the flexing of the spinal column creates a peristaltic fluid motion in the CSF system. In some embodiments the flexing of the spinal column puts pressure on the thecal sac which surrounds the spinal cord and cauda equina. In some embodiments, this flexing is caused by mechanical movement of the spinal column. In some embodiments, this flexing is caused by a device placed external to the body. In some embodiments, an implanted device connected to the CSF system causes pressure gradients within the CSF system resulting in increased CSF flux. In some embodiments, an implanted device pressures the cisterns and/or ventricles of the CSF system to create pressure gradients within the CSF system resulting in increased CSF flux and flow. In an embodiment, the lumbar and cervical areas of the spine are flexed simultaneously.

In an embodiment, an apparatus and method for improving the symptoms of Alzheimer's and other neurodegenerative diseases is disclosed. By increasing CSF flux and flow, which in turn increases the removal of brain and/or CNS waste products, symptoms of Alzheimer's and other neurodegenerative diseases are improved. In embodiments, the neurodegenerative diseases include different forms of dementia including Alzheimer's and Parkinson's and other diseases such as cognitive decline, MCI (mild cognitive impairment), MS (multiple sclerosis), and migraines.

In an embodiment, a method of increasing CSF flux in the area of the spinal column is provided. In an embodiment, a method for improving glymphatic clearance is provided. In an embodiment, increasing CSF flux in the spinal column increases peri-arterial and peri-venous (together perivascular) CSF flux in the brain.

In an embodiment, a method of improving the symptoms of depression is provided. In an embodiment, a method for improving the sleep pattern is provided. In an embodiment, a method for improving memory is provided.

In an embodiment, a method for increasing the speed and/or efficiency and/or effectiveness of delivery of drugs injected into the CSF is provided. In an embodiment, a method for increasing the speed and/or efficiency and/or effectiveness of intravenous chemotherapy drugs for conditions of the brain is provided. In an embodiment, a method for improving the symptoms of chemo-brain is provided.

In an embodiment, a method for improving the symptoms of cognitive decline is provided. In some subjects, increased sagittal vertical axis measurements are correlated with cognitive decline. In an embodiment, the disclosed methods counteract the problems associated with this condition.

In an embodiment, a method for reducing neuroinflammation is provided. Through the use of a CSF pump of the present disclosure, the glymphatic system performance is improved and the effects of injuries, infections, toxins, neurodegenerative diseases, and aging on the brain are decreased.

In an embodiment, a method for increasing the effectiveness of both the glymphatic and lymphatic systems is provided. As discussed herein, waste products from the brain/CNS flow into the CSF and then directly to the lymphatic system.

In an embodiment, an apparatus and method for helping reduce the symptoms of a stroke if provided. Following a stroke, the apparatus and methods for increasing CSF flow and flux will help diminish the damage caused by the stroke.

For many of the embodiments described herein, a method for lowering a subject's brain activity is provided. For many embodiments, when a subject's brain activity is low and brain waves have a lower frequency than beta (alpha, theta, delta) the methods discussed herein are more efficient.

In some embodiments, a method for increasing flow to the glymphatic system comprises positioning a first pump adjacent to a spinal column that is configured to change the curvature of the spinal column. Activating the first pump, changing the curvature of the spinal column and increasing flow to the glymphatic system by increasing the flux of the cerebrospinal fluid (CSF) system. In some embodiments, no external CSF is removed from or added to the CSF system. In some embodiments, the first pump is positioned in the lumbar region of the spinal column and the method further comprises positioning a second pump adjacent the cervical region of the spinal column, activating the second pump and changing the curvature of the cervical region of the spinal column. In some embodiments, the second pump provides flexion or extension. In some embodiments, the method comprises providing electrical, audio, electromagnetic, or mechanical stimulation to the patient. In some embodiments, the first pump is positioned in the backrest of a chair and the first pump comprises a pad configured to apply pressure to the spinal column. In some embodiments, the chair further comprises a headrest and the second pump is positioned to move the headrest so as to provide flexion and extension. In some embodiments, an apparatus is configured to provide oscillating pressure to a patient, positioned on the upper body of the patient so as to increase glymphatic system performance. In some embodiments, the chair is configured to provide a zero-gravity position to the patient.

In some embodiments, a method for reducing neuroinflammation comprises positioning a first pump adjacent to a spinal column that is configured to change the curvature of the spinal column. Activating the first pump, changing the curvature of the spinal column and increasing the flow or flux of the cerebrospinal fluid (CSF) system. In some embodiments, the increased CSF flow or flux improves the performance of the glymphatic system. In some embodiments, an increased level of waste and biological byproducts are removed by the glymphatic system after the pump is activated. In some embodiments, no external CSF is removed from or added to the CSF system. In some embodiments, the first pump is positioned in the lumbar region of the spinal column and the method further comprises positioning a second pump adjacent the cervical region of the spinal column, activating the second pump and changing the curvature of the cervical region of the spinal column. In some embodiments, the second pump provides flexion or extension. In some embodiments, the method further comprises providing electrical, audio, electromagnetic, or mechanical stimulation to the patient. In some embodiments, the first pump is positioned in the backrest of a chair and the first pump comprises a pad configured to apply pressure to the spinal column. In some embodiments, the chair further comprises a headrest and the second pump is positioned to move the headrest so as to provide flexion and extension. In some embodiments, the chair is configured to provide a zero-gravity position to the patient.

In some embodiments, a method for improving symptoms of a stroke comprises positioning a first pump adjacent to a spinal column that is configured to change the curvature of the spinal column. Activating the first pump, changing the curvature of the spinal column and increasing the flow or flux of the cerebrospinal fluid (CSF) system. In some embodiments, the increased CSF flow or flux improves the performance of the glymphatic system. In some embodiments, an increased level of waste and biological byproducts are removed by the glymphatic system after the pump is activated. In some embodiments, no CSF is removed from or added to the CSF system. In some embodiments, the first pump is positioned in the lumbar region of the spinal column and the method further comprises positioning a second pump adjacent the cervical region of the spinal column, activating the second pump and changing the curvature of the cervical region of the spinal column. In some embodiments, the second pump provides flexion or extension. In some embodiments, the method further comprises providing electrical, audio, electromagnetic, or mechanical stimulation to the patient. In some embodiments, the first pump is positioned in the backrest of a chair and the first pump comprises a pad configured to apply pressure to the spinal column. In some embodiments, the chair further comprises a headrest and a second pump is positioned to move the headrest so as to provide flexion and extension. In some embodiments, the chair is configured to provide a zero-gravity position to the patient.

In some embodiments, a method for improving the performance of both the lymphatic and glymphatic systems comprises positioning a first pump adjacent to a spinal column that is configured to change the curvature of the spinal column. Activating the first pump, changing the curvature of the spinal column, and increasing the flow or flux of the cerebrospinal fluid (CSF) system. In some embodiments, the method comprises positioning an apparatus on the body of the subject that is configured to provide mild oscillating pressure to the surface and muscles of the subject, and activating a pressure source to provide the oscillating pressure. In some embodiments, the increased CSF flow or flux improves the performance of the glymphatic system and the oscillating pressure improves the performance of the lymphatic system. In some embodiments, an increased level of waste and biological byproducts are removed by the glymphatic system after the pump is activated. In some embodiments, no CSF is removed from or added to the CSF system. In some embodiments, the first pump is positioned in the lumbar of the spinal column and the method further comprises positioning a second pump adjacent the cervical region of the spinal column, activating the second pump and changing the curvature of the cervical region of the spinal column. In some embodiments, the second pump provides flexion or extension. In some embodiments, the method further comprises providing electrical, audio, electromagnetic, or mechanical stimulation to the patient. In some embodiments, the first pump is positioned in the backrest of a chair and the first pump comprises a pad configured to apply pressure to the spinal column. In some embodiments, the chair further comprises a headrest and the second pump is positioned to move the headrest so as to provide flexion and extension. In some embodiments, the chair is configured to provide a zero-gravity position to the patient.

In some embodiments, a method for improving posture comprises positioning a first pump adjacent to a cervical region of a spinal column that is configured to change the curvature of the spinal column, activating the first pump, changing the curvature of the spinal column and reprogramming muscles to allow for increased range of motion of the muscles.

In some embodiments, a method for improving symptoms of Alzheimer's comprises positioning a first pump adjacent to a spinal column, the pump configured to change the curvature of the spinal column, activating the first pump, changing the curvature of the spinal column and administering antibodies, the antibodies selected to target proteins.

In some embodiments, a therapeutic support device comprises a frame, a first support comprising an aperture, a cerebrospinal fluid pump comprising a body and a pad, the body attached to the frame, the pad extending through the aperture, the pad arranged to move with respect to the first support along an actuation axis between a retracted position and an extended position. In some embodiments, the first support comprises a back support. In some embodiments, the device comprises a second support oriented at an angle to the first support. In some embodiments, the second support comprises a seat. In some embodiments, the second support comprises a headrest. In some embodiments, the second support is arranged to move with respect to the first support. In some embodiments, the second support arranged to rotate with respect to the first support about a rotation axis. In some embodiments, the rotation axis is oriented between the first support and the second support. In some embodiments, the rotation axis oriented orthogonal to the actuation axis. In some embodiments, the device comprises a first orientation wherein the pad is in the retracted position and the second support is oriented at a first angle to the first support. In some embodiments, the device comprises a second orientation wherein the pad is in an intermediate position and the second support is oriented at a second angle to the first support, the second angle being greater than the first angle. In some embodiments, the device comprises a third orientation wherein the pad is in the extended position and the second support is oriented at a third angle to the first support, the third angle being greater than the second angle. In some embodiments, the device comprises a second cerebrospinal fluid pump comprising a second body and a second pad extending through the aperture, the second pad arranged to move with respect to the first support along a second actuation axis. In some embodiments, the actuation axis of a first pump is parallel to the actuation axis of a second pump. In some embodiments, the actuation axis of a first pump is nonparallel to the actuation axis of a second pump. In some embodiments, a range of motion of the first pad is greater than a range of motion of the second pad. In some embodiments, the device comprises a third cerebrospinal fluid pump comprising a third body and a third pad extending through the aperture, the third pad arranged to move with respect to the first support along a third actuation axis. In some embodiments, a therapeutic support device comprises a chair.

In some embodiments, a cerebrospinal fluid pump comprises a base, a pad arranged to move along an actuation axis and a motor arranged to rotate a driveshaft. A first slider is engaged with the driveshaft, which is arranged to move with respect to the base in a first direction upon rotation of the driveshaft. A second slider is engaged with the driveshaft, which is arranged to move with respect to the base in a second direction upon rotation of the driveshaft. A first connector is pivotably attached to the first slider and attached to the pad, and a second connector is pivotally attached to the second slider and attached to the pad. In some embodiments, the first direction is opposite the second direction. In some embodiments, the first slider moves toward the second slider as the driveshaft rotates in a first direction. In some embodiments, the pad moves away from the base as the driveshaft rotates in the first direction. In some embodiments, the first slider moves away from the second slider as the driveshaft rotates in a second direction. In some embodiments, the pad moves toward the base as the driveshaft rotates in the second direction. In some embodiments, the actuation axis is oriented orthogonal to a rotation axis of the driveshaft. In some embodiments, the driveshaft comprises a first portion threaded in a first direction and a second portion threaded in a second direction.

In some embodiments, a cerebrospinal fluid pump comprises a linear actuation device arranged to move a pressure pad along a linear actuation axis. In some embodiments, the linear actuation device comprises a shaft arranged to move along a central axis of the shaft. In some embodiments, the pressure pad is attached to an end of the shaft. In some embodiments, the pressure pad comprises a pad comprising curvature. In some embodiments, the pad comprises a valley. In some embodiments, actuation axis extends through the valley. In some embodiments, the pad comprises a peak positioned beside the valley. In some embodiments, the pad comprises a first peak and a second peak positioned on opposite sides of the valley. In some embodiments, the pressure pad comprises an extension positioned between the shaft and the pad. In some embodiments, multiple extensions of different lengths are provided, which can be used to vary a distance between the linear actuation device and the pad.

In some embodiments, a method for increasing a flow of cerebrospinal fluid (CSF) in a CSF system of a patient comprises applying a pressure to a spinal column region of the patient, thereby changing a curvature of the spinal column. In some embodiments, the method comprises applying the pressure with a pad. In some embodiments, the pad comprises curvature. In some embodiments, the pad comprises a valley. In some embodiments, applying the pressure further comprises displacing the pad in a direction toward the spine. In some embodiments, the method comprises displacing the pad from a first position to a second position. In some embodiments, when the pad is in the first position, the second position overlaps with a position of the spinal column. In some embodiments, when the pad is displaced to the second position, at least a portion of the spinal column is displaced away from the second position. In some embodiments, the curvature of the spinal column is increased as the pad moves to the second position. In some embodiments, the method further comprises retracting the pad in a direction away from the spinal column. In some embodiments, the method further comprises repeating the displacing the pad and retracting the pad. In some embodiments, the displacing of the pad is performed in a first time period and the retracting of the pad is performed in a second time period. In some embodiments, second time period is greater than the first time period. In some embodiments, the spinal column comprises a lumbar region and a cervical region, and applying the pressure comprises applying pressure to the lumbar region, thereby changing a curvature of the lumbar region. In some embodiments, the method further comprises moving a head of the patient to change a curvature of the cervical region. In some embodiments, the method comprises increasing the curvature of the lumbar region while moving the head to provide flexion of the cervical region. In some embodiments, the method comprises reducing the curvature of the lumbar region while moving the head to provide extension of the cervical region. In some embodiments, the moving of the head to provide flexion of the cervical region pad is performed in a first time period and the moving of the head to provide extension of the cervical region is performed in a second time period. In some embodiments, the second time period is longer than the first time period. In some embodiments, the method comprises providing a cerebrospinal fluid pump comprising a pad and applying the pressure with the pad. In some embodiments, the method comprises controlling the cerebrospinal fluid pump to displace the pad in a direction toward the spinal column. In some embodiments, the method comprises controlling the cerebrospinal fluid pump to retract the pad in a direction away from the spinal column. In some embodiments, the method comprises providing a second cerebrospinal fluid pump comprising a second pad and applying a pressure to the spinal column region with the second pad. In some embodiments, the first cerebrospinal fluid pump is offset from the second cerebrospinal fluid pump.

These and other embodiments which characterize the invention are pointed out with particularity in the claims annexed hereto and forming a part hereof. However, for a better understanding of the invention, its advantages and objectives obtained by its use, reference can be made to the drawings which form a further part hereof and the accompanying descriptive matter, in which there are illustrated and described various embodiments of the invention.

### BRIEF DESCRIPTION OF THE DRAWINGS

FIGS. 1A, 1B, and 1C are schematic diagrams of the CSF system.

FIG. 2 is a schematic diagram of the glymphatic system.

FIGS. 3A-3C show views of an embodiment of a schematic diagram of a CSF pump.

FIG. 4 shows an embodiment of a schematic diagram of a CSF pump.

FIG. 5 shows an embodiment of a schematic diagram of a CSF pump.

FIGS. 6A and 6B are two views of a schematic diagram of a CSF pump mounted on a chair.

FIGS. 7A and 7B are schematic diagrams of implantable CFS pumps.

FIG. 8 is a schematic diagram of a CSF pump mounted on a chair.

FIGS. 9A and 9B show flexure of the spinal column.

FIGS. 10A and 10B are diagrams of a CSF pump mounted on a chair and a second pump associated with a moveable headrest.

FIGS. 11A, 11B, and 11C show and embodiment of a CSF pump chair and flexure of the spinal column.

FIG. 12 is an embodiment of a schematic diagram of a CSF pump.

### DETAILED DESCRIPTION

The present disclosure includes methods and apparatuses for increasing cerebrospinal fluid (CSF) flux. An example apparatus includes a pump configured to change the dynamics of the CSF system. In one example, the apparatus includes a pump configured to alternatingly provide and release pressure on the spinal column and to allow the spinal column to flex and to move back to the unpressured position. This pressure causes spinal column flexing or change of curvature and a resulting change in CSF fluid dynamics. In some embodiments the pump is positioned outside a subject's body. In some embodiments the pump is an implanted device that causes a pressure wave or changes the pressure in the CSF system that changes the CSF system dynamics. In some embodiments, the changed CSF dynamics and resulting increased CSF flux results in improving the symptoms of Alzheimer's and/or other cognitive diseases.

In the following detailed description of the present disclosure, reference is made to the accompanying drawings that form a part hereof, and in which is shown by way of illustration how one or more embodiments of the disclosure may be practiced. These embodiments are described in sufficient detail to enable those of ordinary skill in the art to practice the embodiments of this disclosure, and it is to be understood that other embodiments may be utilized and that process, electrical, and structural changes may be made without departing from the scope of the present disclosure.

As used herein, designators such as “X”, “Y”, “N”, “M”, etc., particularly with respect to reference numerals in the drawings, indicate that a number of the particular feature so designated can be included. It is also to be understood that the terminology used herein is for the purpose of describing particular embodiments only and is not intended to be limiting. As used herein, the singular forms “a”, “an”, and “the” can include both singular and plural referents, unless the context clearly dictates otherwise. In addition, “a number of”, “at least one”, and “one or more” (e.g., a number of pivot points) can refer to one or more pivot points, whereas a “plurality of” is intended to refer to more than one of such things. Furthermore, the words “can” and “may” are used throughout this application in a permissive sense (i.e., having the potential to, being able to), not in a mandatory sense (i.e., must). The term “include,” and derivations thereof, means “including, but not limited to”. The terms “coupled” and “coupling” mean to be directly or indirectly connected physically or for access to and movement of the movable handle member, as appropriate to the context.

The mechanisms behind CSF dynamics in humans are still not fully understood. CSF circulates from its primary production sites at the choroid plexus through the brain ventricles to reach the outer surface of the brain in the subarachnoid spaces from where it drains into the venous bloodstream and cervical lymphatics. According to one concept of brain fluid transport, fluid enters the brain across the blood/brain barrier and from the CSF system. Fluid exits the brain through the arachnoid villa surface also enters the brain tissue along peri-arterial routes and exits through peri-venous routes again subarachnoid compartments. Fluid also flows directly from both the ISF system and CSF system to the lymphatic system.

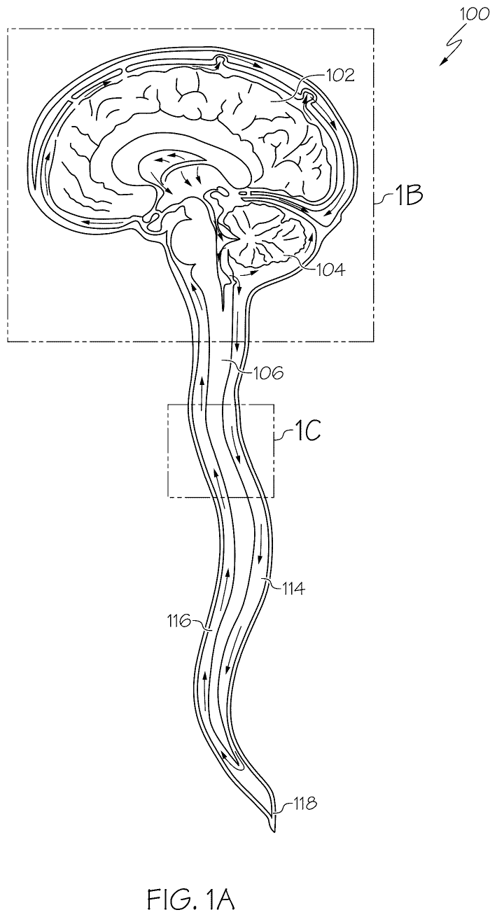

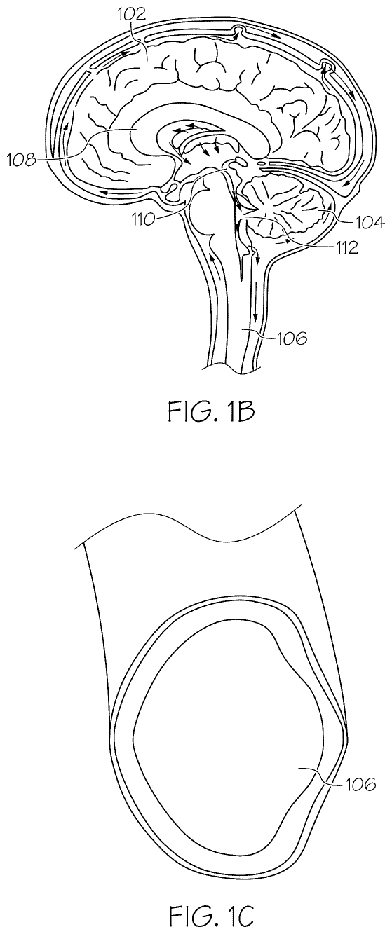

FIGS. 1A, 1B, and 1C are schematic diagrams showing the flow of CSF in the human body. CSF is produced mainly by the choroid plexus epithelium and ependymal cells of the ventricles. Shown in FIG. 1B is one of the lateral ventricles 108 (as this is a side view the other lateral ventricle is not visible), the third ventricle 110, and the fourth ventricle 112. From the primary site of secretion in the choroid plexus, CSF flows throughout the ventricular system of the brain. CSF flows from the lateral ventricles 108 to the third ventricle 110 via the foramen of Monro. From here, it flows across the cerebral aqueduct of Sylvius to the fourth ventricle. From the fourth ventricle CSF flows into the dorsal spinal subarachnoid space through the foramen of Magendie and into the basal cisterns through the lateral foramina of Luschka. Passage of CSF through the foramen of Magendie into the vallecula and the beginning of downward flow into the dorsal cervical subarachnoid space precedes the exit of CSF from the foramina of Luschka. CSF then flows caudally through the dorsal spinal subarachnoid space 114 and into the lumbar cistern 118 followed by ascent of CSF in the ventral spinal subarachnoid space 116.

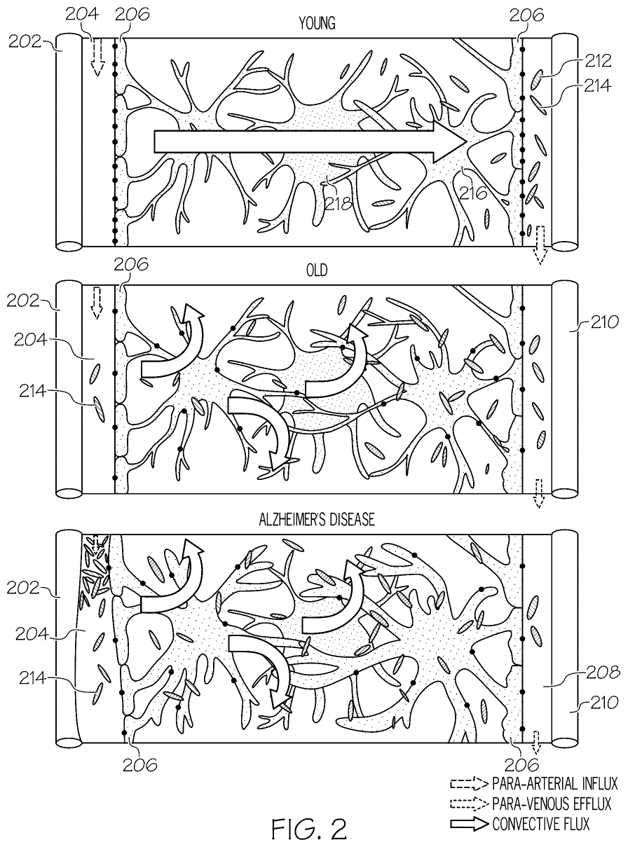

In the interstitial space in the brain, the CSF system clears out toxic aggregates, toxins and cellular waste products. These can include tau and beta amyloids. The accumulation of toxins and waste products are seen to accumulate in association with neurodegenerative and neurotoxic diseases such as Alzheimer's disease. It is believed that if these toxins and waste products can be removed a subject's symptoms would improve. As shown in FIG. 2, CSF travels to the brain in the peri-arterial space 204 that surrounds the arteries 202. Fluid and nutrients from CSF flow to the interstitial space which contains neurons 218 and astrocytes 216. Water channels called aquaporin 4 (AQP4) 206 facilitate the flow of this fluid from the peri-arterial space 204 and into the peri-venous space 208 that surrounds the veins 210 in the brain. As shown in FIG. 2, in the glymphatic system fluid enters the interstitial space of the brain from the peri-arterial channels 204. It moves across the astrocytes 216 and/or neurons 218 and ‘washes’ toxins and cellular waste products such as metabolic waste 212 and beta-amyloid 214 through AQP4 206 and into the peri-venous channels 208.

While the CSF system does remove toxins and waste products, some amount of beta-amyloid 214 and other waste products may be present in the CSF supply in the peri-arterial space 204. Some studies have shown that following traumatic brain injury or axon damage, the concentration of tau proteins dramatically increases in the CSF.

Increased flux of CSF in the CNS system will improve the flux and flow of CSF in the glymphatic system, increase the efficiency of the glymphatic system, and increase the glymphatic clearance. As described herein, changing the dynamics of the CSF system will result in increased flux and flow of the CSF system. This increased flux and flow will increase the filtering and generation of CSF by the body. The increased flux and pressure, along with increased CSF generation all will contribute to higher peri-arterial CSF flux. Higher peri-arterial flow of CSF will result in higher peri-venous return flow.

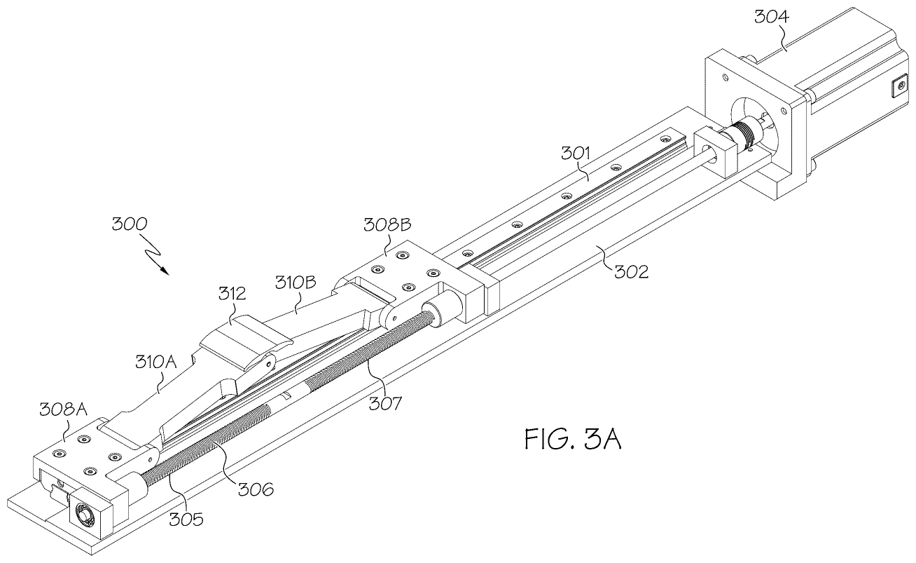

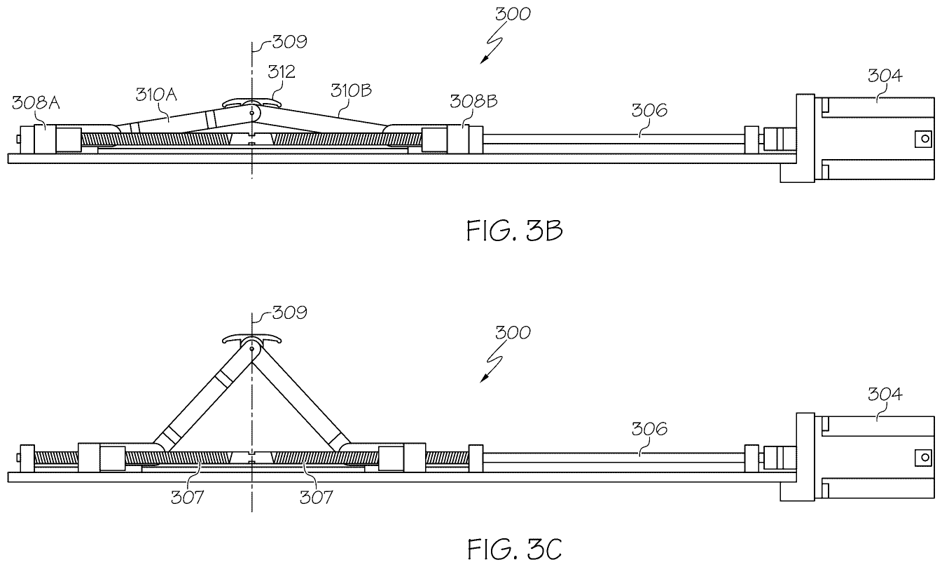

FIGS. 3A-3C show an embodiment of a schematic diagram of a CSF pump 300 that is placed external to the body. Pump 300 includes base 302 that supports a track 301. Extending along the base is rotating shaft 306, attached at one end to motor 304. Shaft 306 is also connected to a pair of sliders, 308A and 308B. Sliders 308A and 308B are configured to slide with respect to base 302 along track 301 without becoming disconnected from the track 301. As shaft 306 rotates in a first direction, sliders 308A and 308B will move toward each other. When shaft 306 rotates in a second direction, sliders 308A and 308B will move away from each other. First connector 310A is pivotably connected to first slider 308A at one end and pad 312 at the other end. The connection for arm 308A to pad 312 also permits pivoting. Second connector 310B is pivotably connected to slider 308B at one end and pad 312 at the other end. The connection for arm 308B to pad 312 also permits pivoting. As motor 304 rotates shaft 306 in a first direction, sliders 308A and 308B will move toward each other causing pad 312 to move away from base 302, for example along an actuation axis 309. As motor 304 rotates shaft 306 in a second direction, sliders 308A and. 308B move away from each other causing pad 312 to move toward the base. In some embodiments, shaft 306 comprises threads that interface with threads on sliders 308A and 308B to provide the motion of the pad 312. In some embodiments, a pad 312 can have any suitable shape and is desirably arranged to apply pressure to a human body. In some embodiments, a pad 312 comprises curvature. In some embodiments, shaft 306 comprises opposite threads such that threads in one direction interact with slider 308A and threads in the opposite or second direction interact with slider 308B. In some embodiments, shaft 306 comprises a first portion 305 and a second portion 307. In some embodiments, the first portion 305 comprises first threads and the second portion 307 comprises second threads. In some embodiments, the first threads comprise forward threads and the second threads comprise reverse threads. In some embodiments, an actuation axis 309 is oriented orthogonal to a central axis of the shaft 306.

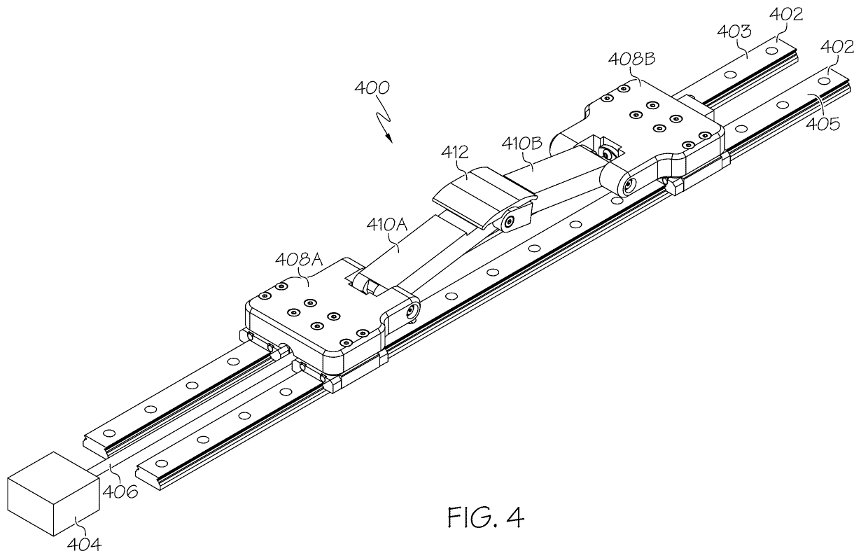

FIG. 4 shows an embodiment of a schematic diagram of a CSF pump 400 that is placed external to the body. Pump 400 includes base 402. In this embodiment, base 402 comprises two pieces which may or may not be attached to each other. In some embodiments, the base 402 comprises a first track 403 and a second track 405. Extending along the base is rotating shaft 406, attached at one end to motor 304. Shaft 307 is also connected to a pair of sliders, 308A and 308B. Sliders 308A and 308B are configured to slide along base 302 without becoming disconnected from the base. In some embodiments, each slider 308A, 308B is attached to the first track 403 and to the second track 405. As shaft 306 rotates in a first direction, sliders 308A and 308B will move toward each other. When shaft 306 rotates in a second direction, sliders 308A and 308B will move away from each other. Slider 308A is pivotably connected to slider 308A at one end and pad 312 at the other end. The connection for arm 308A to pad 312 also permits pivoting. Slider 308B is pivotably connected to slider 308B at one end and pad 312 at the other end. The connection for arm 308B to pad 312 also permits pivoting. As motor 304 rotates shaft 306 in a first direction, pads 308A and 308B will move toward each other causing pad 312 to move away from base 302. As motor 304 rotates shaft 306 in a second direction, pads 308A and. 308B move away from each other causing pad 312 to move toward the base.

The pumps of FIGS. 3 and 4 can be used in situations where space behind a subject's spinal column is minimal. For example, the pumps shown could be used with a standard home or hospital bed. These pumps can be constructed such that very little space is needed between the surface below (in some embodiments the top of a bed) and the posterior surface of a subject's back. In some embodiments, 3 to 6 cm. In some embodiments where there is more space or where a specialized chair or bed or the like will be constructed, an electric cylinder (or electro-mechanical cylinder) pump can be used. Electric cylinders convert rotary mechanical force into linear motion as is known in the art. While electric cylinders are described, pneumatic cylinders, which translate pressurized flow into kinetic motion, usually with a piston or slide, can also be used. For any of the pumps shown herein, airbags or a fluid filled bag could be used. Such airbag or fluid filled bag could also be used to move the pressure pads.

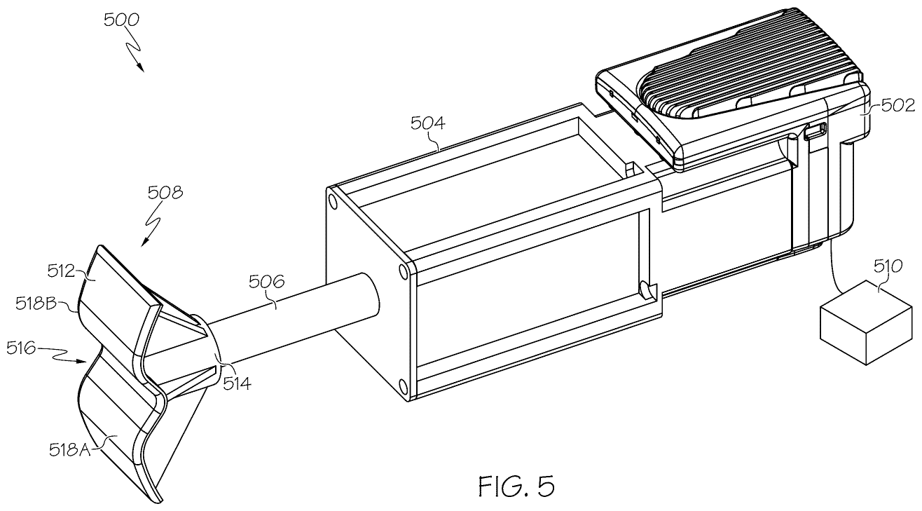

Shown in FIG. 5 is cylinder pump 500. In some embodiments, cylinder pump 500 comprises a linear actuator. Cylinder pump 500 comprises base 502. Base 502 includes the power source (motor) for the pump and may include a control system to allow for programming of the pump. Attached to the base 502 is cylinder 504, which works with base 502 to provide axial movement of shaft 506 of cylinder 540. Shaft 506 extends from retracts into cylinder 504 to provide the pump motion. Attached to the end of shaft 506 is pressure pad 508. In some embodiments, pressure pad 508 comprises pad 512 and extension 514. In some embodiments, a pad 512 comprises curvature. In some embodiments, a pad 512 comprises a valley 516 or recess oriented between a first peak 518A and a second peak 518B. In some embodiments, the valley 516 is centrally located in the pad 512. In some embodiments, the valley 516 is positioned over the shaft 506. In some embodiments, the valley 516 helps to avoid applying pressure to central portions of the spinal column, such as the spinous process of vertebra. In various embodiments, the pad 512 shape shown in FIG. 5 can be used with any type of cerebrospinal fluid pump. In some embodiments (and for any of the pumps/devices shown herein), multiple pressure pads 508 are provided with different lengths of extension 514. This allows a user of the pump to select a pressure pad with a length that allows the pressure pad to fit snugly to the back of a patient without the need to extend or retract shaft 506. In some embodiments, pump 500 includes a user interface 510, electrically or wirelessly connected to pump 500. In some embodiments, interface 510 includes the control system for pump 500.

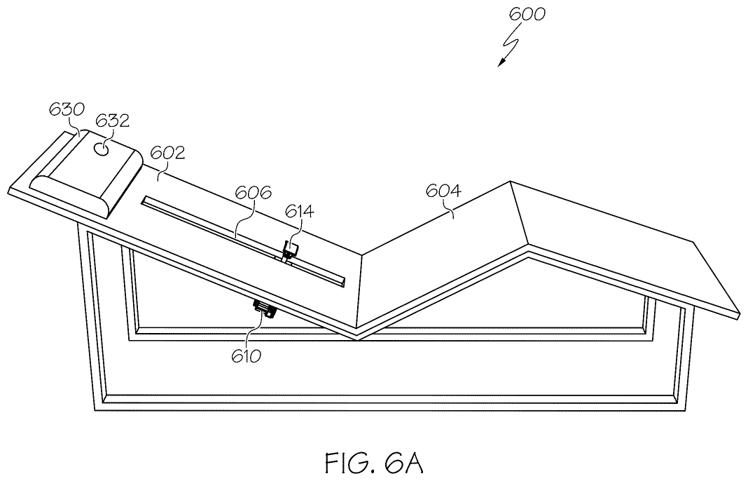

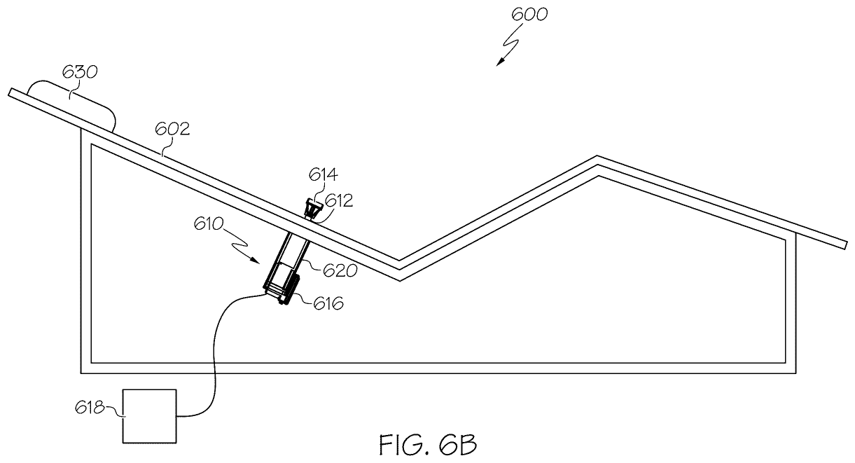

FIGS. 6A and 6B are views of an embodiment of a chair fitted with a cylinder pump, although pumps as shown in FIGS. 3 and 4 could also be used. A perspective view of chair 600 is shown in FIG. 6A. Shown is back rest 602 and seat 604. Positioned in back rest 602 is opening 606. In some embodiments, the opening 606 is centered across a width of the back rest 602. In some embodiments, the pump is centered across a width of the back rest 602. Pump pressure pad 614 is positioned within opening 606. While chair 600 is shown reclined at an angle, it could also be upright. Pressure pad 614 is shown to be about at the top surface of back rest 602. When used, pressure pad 614 will be positioned near the top of or above back rest 602 at a height where is touches the posterior skin/clothing surface of a subject's spinal column. An optional headrest 632 is provided in some embodiments. In further embodiments, and energy delivery port 632 is associated with the head rest 630. A side view of chair 600 is shown in FIG. 6B. In this view, cylinder pump 610 is shown. Pump 610 comprises base/power source/motor 616, cylinder 620, shaft 612 pressure pad 614, and optional user interface 618 and/or headrest 630. In some embodiments, movement of pad 614 can be described as being generally perpendicular to the plane of back rest 602. In embodiments where back rest 602 is curved, this plane is generally in the area where pad 614 is positioned.

In use, a subject, not shown, will be positioned in chair 600. The physician/technician will use a pressure pad with an appropriate length or will program pump so that pressure pad 614 is moved into contact with the clothing/skin of the subject in the posterior area of the spinal column. Cylinder pump 610 can be positioned within opening 606 so that the proper section of the subject's spinal column is positioned adjacent to pressure pad 614. The physician/technician will program the pump with information such as length of stroke and speed of stroke. In some embodiments, the length of stroke is 1 to 5 cm, 2 to 4 cm, or is 1, 2, 3, 4, or 5 cm and the speed is set so that each extension and retraction is 1 to 10 or 4 to 10 seconds. The extension and retraction may be set to different speeds. In some embodiments, the extension corresponds to a subject's inhalation and the retraction corresponds to a subject's exhalation (the movement of the pressure pad is gated to the respiratory cycle). When activated, the pressure pad will extend the desired distance and flex or change the curvature of the spinal column. This flexing of the spinal column will cause a change in the dynamics of the CSF system. In some embodiments, no external CSF is added to the CSF system. In some embodiments, no CSF is separately removed from the CSF system. In some embodiments, the flexing of the spinal column increases the hydrostatic pressure of the CSF which results in increased CSF flow and flux. In some embodiments, a subject's breathing pattern will automatically switch to match the pump motion—with the patient inhaling on pump extension and exhaling with pump retraction. In some embodiments, a sensor, not shown, is positioned to sense a subject's breathing pattern and the pump is programmed to match the patient's breathing pattern. In some embodiments, more than one pump may be used. In this embodiment, the stroke of the one of more cylinder pumps may be different. In some examples, the cylinder pump in the center may have the longest stroke (to cause the most flexing). In some multiple pump examples, instead of individual pressure pads, one long flexible pad may be used.

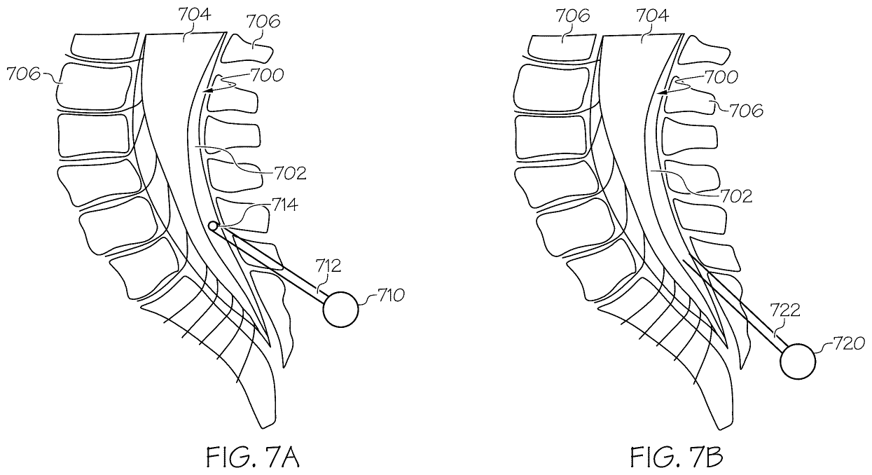

While some embodiments show a pump positioned external to a subject's body, and implantable pump can also be used. In another embodiment, a pump or device can put pressure on or inside one of the CSF ventricles or cisterns. To put pressure on the ventricle or cistern, a pump configured to introduce a pressure gradient. FIGS. 7A and 7B show embodiments of pumps that are used to directly pressure the CSF system. In 7A, a small bladder 714 is surgically implanted in a ventricle or cistern of the CSF system and attached to pump 710 via tube 712. In FIG. 7A, the bladder is positioned in lumbar cistern 700 although other parts of the CSF system can be used. Also shown in spinal cord 714, vertebral column 706, CSF 702. Pump 710 can include a power source/motor, controller, receiver, and a pump for moving a fluid (air, water, saline, etc.) in and out of bladder 714. The repeated pressurizing/depressurizing of bladder 714 will create pressure waves within the CSF 702 and change the dynamics of the CSF system. These changed dynamics will result in increased CSF flux. Pump 710 may include a replaceable or rechargeable battery or may receive power wirelessly from a power source positioned outside a subject's body (not shown). The receiver in pump 710 will allow a physician/surgeon to program the pump's operating conditions. FIG. 7B shows an embodiment where a pipette- or dropper-like device 720 is implanted in fluid communication with a CSF ventricle or cistern and filled with CSF via tube 722. The pump 720 is configured to pressurize/depressurize the pipette or dropper to cause the CSF to flow into and out of the ventricle or cistern. In FIG. 7B, the end of tube 622 is positioned in lumbar cistern 700 although other parts of the CSF system can be used. Also shown in spinal cord 714, vertebral column 706, CSF 702. Pump 720 can include a power source, controller, receiver, and a pump for moving a fluid (air, water, saline, etc.) in and out of lumbar cistern 700. This in and out flow of CSF will impart a pressure wave into the CSF system to change the dynamics of the CSF system. While both pumps 710 and 720 are shown in conjunction with the lumbar cistern 700, any opening if the CSF system, including a cistern or ventricle can be used.

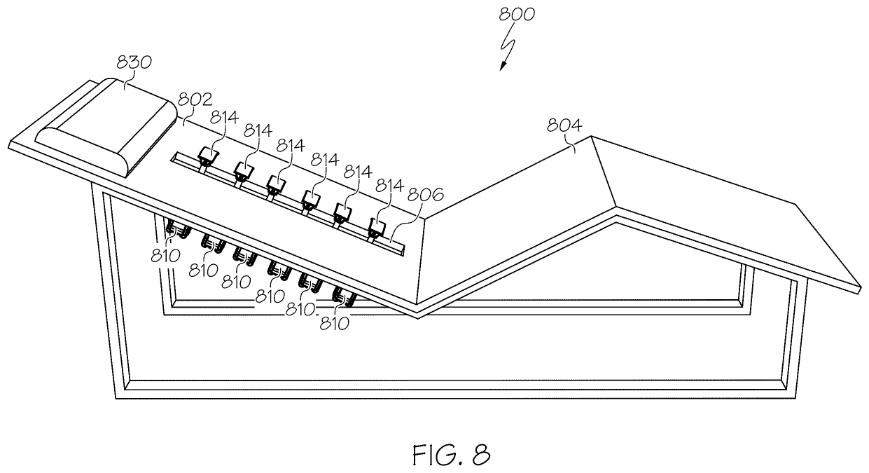

FIG. 8 is a perspective view of a chair fitted with multiple cylinder pumps. Shown is back rest 802 and seat 804. Positioned in back rest 802 is opening 806. Pump pressure pads 814 is positioned within opening 806. Positioned under back rest 802 are corresponding pumps 810. While chair 800 is shown reclined at an angle, it could also be upright. Pressure pads 14 is shown to be about at or slightly above the top surface of back rest 802. When used, pressure pads 814 will be positioned near the top of or above back rest 802 at a height where they touch the posterior skin/clothing surface of a subject's spinal column. An optional headrest 830 is provided in some embodiments. In some embodiments, movement of pads 814 can be described as being generally perpendicular to the plane of back rest 802. In embodiments where back rest 802 is curved, this plane is generally in the area where pad 814 is positioned. While the figure shows six pumps, any number from one to six or more could be used. While individual pressure pads 814 are shown, one pressure pad that covers all the pumps could be used. When in use, a subject will be positioned in chair 800 with the posterior of the subject's body closest to pumps 810. Pumps 810 can be programmed to raise pads 814 all at the same time and for the same distance or at different times and/or for different differences. For example, the pumps could be sequences so that the pump closes to the end of the spinal column raises its pad 814 first, followed by the next pump further up the spinal column, and so on. Alternatively, the pump nearest the top of the spinal column could go first. Multiple adjacent pumps can have any suitable orientation. In some embodiments, an actuation axis of a first pump is parallel to an actuation axis of a second pump. In some embodiments, an actuation axis of a first pump is nonparallel to an actuation axis of a second pump.

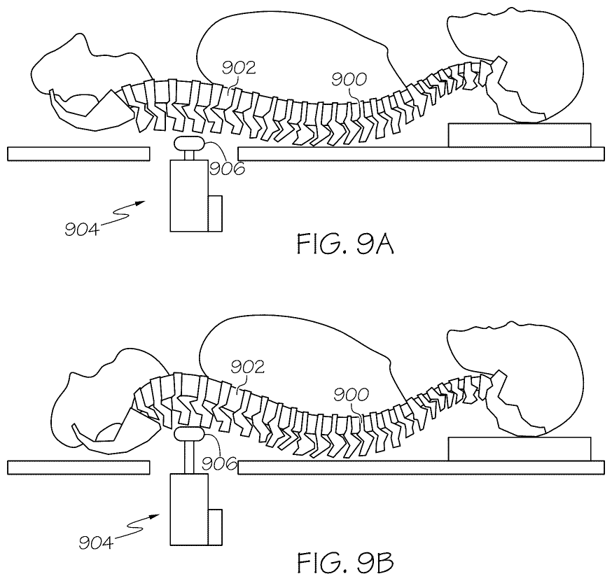

FIGS. 9A and 9B are a diagram of a pump of the current invention 904 and pressure pad 906 positioned adjacent to a subject's spinal column 900. When pressure pad is not applying sufficient pressure to flex spinal column, as shown in FIG. 9A, the curvature of the spinal column at the position directly over pressure pad 906 is normal or at rest. When pump 904 and pressure pad 906 apply more pressure to spinal column 900, the curvature of the spinal column at the position directly over pressure pad 906 is flexed or has more curvature as compared to the at rest position. This flexing or change of curvature and a resulting change in CSF fluid dynamics. In some embodiments, the flexing causes pressure to be applied to the CSF in the thecal sac surrounding the spinal cord and the cauda equina.

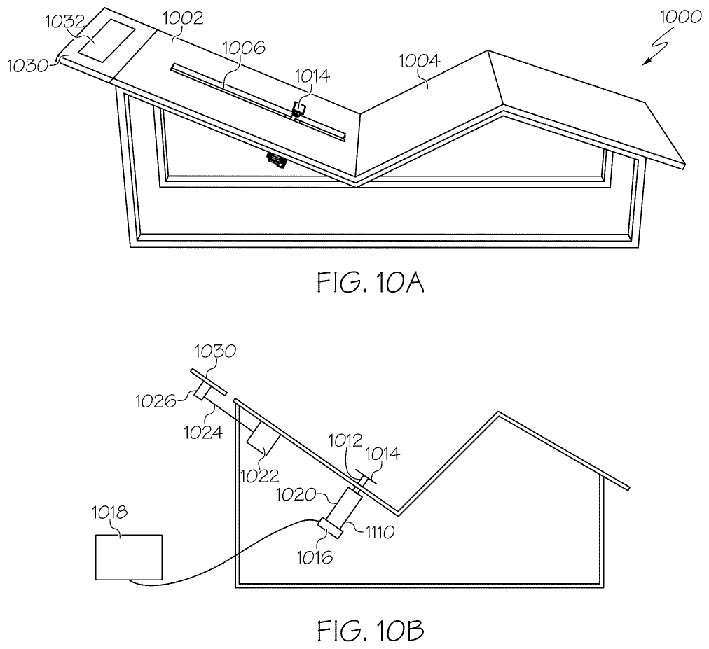

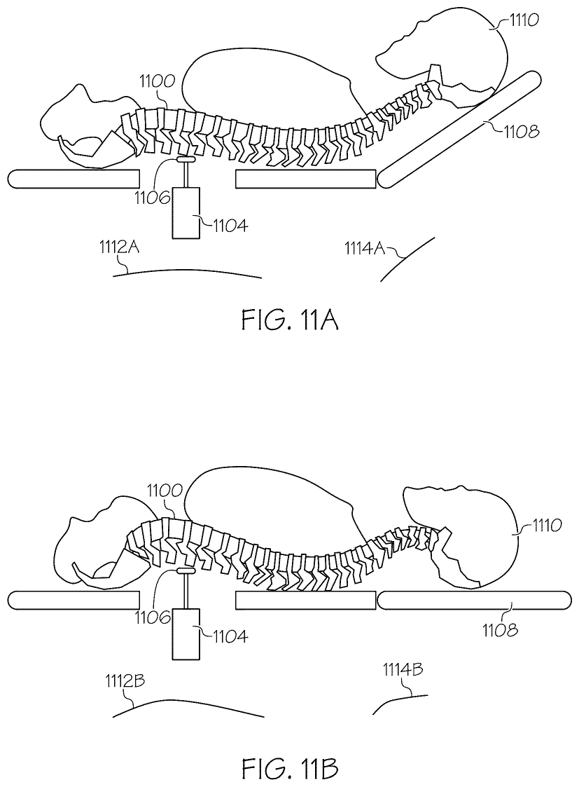

FIGS. 10A and 10B are views of a chair fitted with a cylinder pump, although pumps as shown in FIG. 3, 4, or 12 could also be used. A perspective view of chair 1000 is shown in FIG. 10A. Shown is back rest 1002 and seat 1004. Positioned in back rest 1002 is opening 1006. Pump pressure pad 1014 is positioned within opening 1006. Moveable headrest 1032 is positioned at the top of back rest 1002. In some embodiments, headrest 1032 is configured in a rotational fashion such that, when activated, it provides lateral flexion and/or rotation to a subject using the chair. In some embodiments, headrest 1032 is configured to allow flexion and/or extension to a subject using the chair. While chair 1000 is shown reclined at an angle, it could also be upright. Pressure pad 1014 is shown to be about at the top surface of back rest 1002. When used, pressure pad 1014 will be positioned near the top of or above back rest 1002 at a height where is touches the posterior skin/clothing surface of a subject's spinal column. In further embodiments, and energy delivery port 1032 is associated with the head rest 1030. A side view of chair 1000 is shown in FIG. 10B. In this view, cylinder pump 1010 is shown. Pump 1010 comprises base/power source/motor 1016, cylinder 1020, shaft 1012 pressure pad 1014, and optional user interface 1018 and moveable headrest 1030. In some embodiments, movement of pad 1014 can be described as being generally perpendicular to the plane of back rest 1002. Electric cylinder/actuator, or other movement inducing device, 1022 is pivotably attached to head rest 1032 at 1026 via shaft 1024. As electric cylinder/actuator 1022 extends shaft 1024, head rest 1032 will rotate in an upward direction. When electric cylinder/actuator 1022 retracts shaft 1024, head rest 1032 will rotate in a downward direction. In embodiments where back rest 1002 is curved, this plane is generally in the area where pad 1014 is positioned. In some embodiments, as pump 1010 provides pressure to the lumbar and/or thoracic regions of the spinal column, head rest 1032 moves downward to provide an extension motion to the subject's head and move upward to provide flexure to the cervical area of the spinal column. When pump 1010 moves in a downward direction to lessen the pressure being put on the lumbar and/or thoracic sections of the spinal column, headrest 1030 moves in an upward direction to provide a flexion movement to a subject's head, providing flexure to the cervical area of the spinal column.

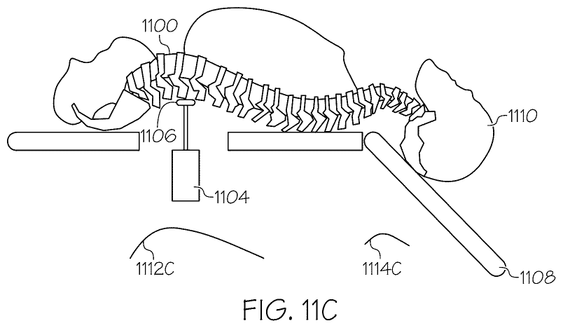

FIGS. 11A, 11B, and 11C are a diagram of an example of a pump of the current invention 1104 and pressure pad 1106 positioned adjacent to a subject's spinal column 1100 and a moveable headrest 1108 positioned under a subject's head 1110. When pressure pad is not applying sufficient pressure to flex spinal column, as shown in FIG. 11A, the curvature of the spinal column at the position directly over pressure pad 1106 is normal or at rest and headrest 1108 is positioned in an upward position providing flexion to a subject's head. Curve 1112A is an approximation of the curve of the spinal column directly above pressure pad 1106 and curve 1114A is an approximation of the curve in the cervical spine adjacent to head 1110. As shown in FIGS. 11B, when pump 1104 and pressure pad 1106 apply pressure to spinal column 1100, the curvature of the spinal column at the position directly over pressure pad 1106 begins to flex or has more curvature as compared to the at rest position and headrest 1108 is positioned in a downward position transitioning from flexion to extension of a subject's head and flexing to the cervical region of the spinal column. Curve 1112B is an approximation of the curve of the spinal column directly above pressure pad 1106 and curve 1114B is an approximation of the curve in the cervical spine adjacent to head 1110. As shown in FIGS. 11C, when pump 1104 and pressure pad 1106 have been completely extended, the curvature of the spinal column at the position directly over pressure pad 1106 has more curvature as compared to what is shown in FIGS. 11A and 11B and headrest 1108 is positioned in a downward position providing extension to a subject's head and flexing to the cervical region of the spinal column. Curve 1112C is an approximation of the curve of the spinal column directly above pressure pad 1106 and curve 1114C is an approximation of the curve in the cervical spine adjacent to head 1110. This flexing or change of curvature of the lumbar/thoracic and cervical regions results in a change in CSF fluid dynamics. In some embodiments, the flexing causes pressure to be applied to the CSF in the thecal sac surrounding the spinal cord and the cauda equina. In some embodiments, a therapeutic support device comprises a first orientation, for example as shown in FIG. 11A. In some embodiments, in the first orientation, the headrest 1108 is oriented at a first angle to the backrest and the pressure pad 1106 is oriented in a retracted position, for example being closest to the backrest along its range of travel. In some embodiments, a therapeutic support device comprises a second orientation, for example as shown in FIG. 11B. In some embodiments, in the second orientation, the headrest 1108 is oriented at a second angle to the backrest and the pressure pad 1106 is oriented in an intermediate position, for example being midway along its range of travel. In some embodiments, a therapeutic support device comprises a third orientation, for example as shown in FIG. 11C. In some embodiments, in the third orientation, the headrest 1108 is oriented at a third angle to the backrest and the pressure pad 1106 is oriented in an extended position, for example being farthest from the backrest along its range of travel. In some embodiments, a movement from the first orientation to the third orientation is performed in a first time period, and the return movement from the third orientation back to the first orientation is performed in a second time period. In some embodiments, the second time period is greater than the first time period.

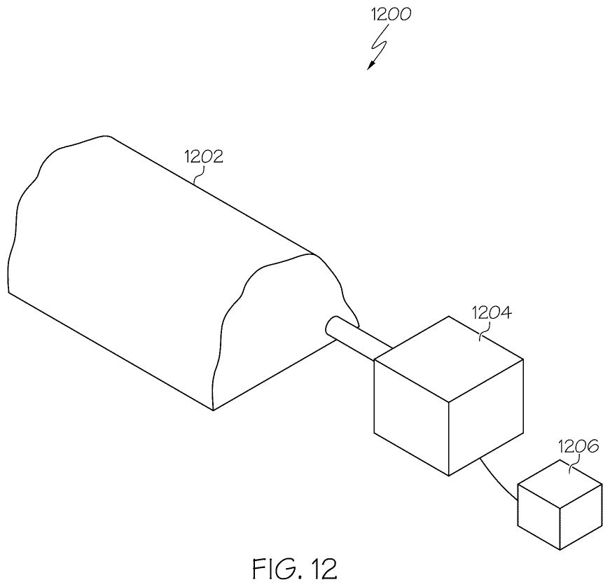

FIG. 12 is a diagram of a pump 1200 that uses a semi- or non-compliant bag 1202 to provide pressure to the spinal column. Any fluid, gas or liquid, can be used to inflate bag 1202. Shown is fluid pump 1204 connected to bag 1202. Controller 1206 controls pump 1204 to provide inflation and deflation to bag 1202. While bag 1202 is shown with the shape of a rounded triangle prism, other prism or pyramid shapes can be used.

The present invention provides a method of improving CSF flux. The method includes positioning a pump near a subject's spinal column on the dorsal or posterior side. In some embodiments, the pump does not require any internal access, through the skin of the patient (either temporary or permanent access). In some embodiments the pump is positioned to act on L2 and/or L3 but can be positioned anywhere on the spinal column. While the pump may put pressure on only a part of the spinal column, a larger segment of the spinal column will flex. The method includes putting pressure on or moving the spinal column in an anterior direction. In some embodiments, movement of 5 mm is sufficient. In some embodiments, movement of 5 to 20 mm, 5 to 15 mm, 5 to 10 mm, 10 to 20 mm, 5 mm, 10 mm, 15 mm, 20 mm, 25 mm, 30 mm, 35 mm, 40 mm, 45 mm, 50 mm, 55 mm, 60 mm, 65 mm, 75 mm, or more is provided. As the spinal column flexes in response to the pressure, CSF flux is increased. This flexing or spinal cord movement results in increased CSF flux. In some embodiments, the flexing or changing the curvature of the spinal column results in pressure being applied to the CSF in the thecal sac surrounding the spinal cord and the cauda equina. In some embodiments, the flexing of the spinal column results in the spinal cord and the CSF flow chambers to act as a peristaltic pump in pumping the CSF. In some embodiments, it is increased in both the caudal or downward and cephalad or upward directions. In some embodiments, the movement if provided in a rhythmic fashion, for example moved anteriorly for 5 seconds and posteriorly (allowed to return to the normal position) for 5 seconds. In other examples, other durations such as 1 to 10, 3 to 8, 5 to 10, 2, 4, 6, 8, 10, 12, 14 or more seconds are used. In some embodiments, the posterior and anterior movements are not at the same speed or for the same duration. In some embodiments, anterior movement is performed in a first time period and posterior movement is performed in a second time period, wherein the second time period can be generally greater than the first time period. In some embodiments, the pump is electrically or wirelessly connected to a sensor for sensing the breathing pattern of the subject. In this embodiment, the pump moves anteriorly during an inhalation and posteriorly in exhalation. In some embodiments, a subject's breathing pattern will gate with the pump movement even in the absence of a sensor. In some embodiments, the treatment has a duration of 10 min, 15 min, 20 min, 25 min, 30 min, or more.

In some embodiments, the CSF pump in configured to change the spinal vertebral curvature. This curvature includes the cervical lordotic curve, the thoracic kyphotic curve, the lumbar lordotic curve, the sacral curve, and/or the coccygeal curve. In some embodiments, the pressure pad of the pump has a length that extends for a distance of 1 to 3, 1 to 2, 1, 2, 3, 5, 10, 15, or more vertebra. In some embodiments, an auxiliary pad of the same length is used with the pump's pad. In some embodiments, this long pad is constructed of spring steel or other material that transmits force but is flexible. The long pad is positioned along the spine and over the pump. When the pump puts pressure on the spinal column, the long pad helps distribute the force of the pump and can be more comfortable to the subject. When using a pad that extends over multiple vertebrae, it is possible to use a pump system with multiple pumps and/or pressure pads. In some embodiments of the multiple pad pump system, the various pads may move the spinal column in an anterior direction in different amounts. For example, in an embodiment with three pads, the middle pad may provide spinal column movement that is larger than the two outside pads. In some embodiments, a soft pad, such as a foam material, may be positioned on top of the pressure pad to make the treatment more comfortable for the subject.

In some embodiments, in an effort to show changes in the dynamics of the CSF system, magnetic resonance imaging (MRI) of parts of the CSF system, as is known in the art, is used. Quantitative CSF flow can also be measured by PC MRI (phase contrast magnetic resonance imaging).

In some embodiments, the present invention provides a method for improving glymphatic clearance. As discussed above, in the glymphatic system, fluid from the CSF flows from the peri-arterial space, over the neurons and astrocytes, and back into the CSF of the peri-venous space. By increasing flux of the CSF in the area of the spine, there is increased flow of CSF through the glymphatic system. This results in increased filtering of the CSF resulting in less metabolic waste products in the CSF and in the CSF in the peri-arterial space. This allows the glymphatic system to clear away more metabolic waste products such as beta-amyloids during periods of increased CSF flux.

It is believed that the CSF system, especially the removal of toxic aggregates and metabolic waste, including beta amyloid, from the brain (glymphatic clearance) works best when a subject's brain activity is low and brain waves have a lower frequency than beta (alpha, theta, delta) which normally occurs during sleep or mediation or a similar mental state. During low brain activity such as sleep, blood pressure drops which allows the peri-arterial and peri-venous channels that carry CSF to the brain to open more, resulting in increased CSF flow. In some embodiments, methods can be used to help a patient relax and be in a state that is sleep-like. In some examples, energy port 632 can be used to provide electro/mechanical energy (EME) to the subject. This EME can be in the form of electrical, electromechanical, electromagnetic, mechanical, sound, audio, pressure, visual, visible or non-visible electromagnetic radiation, or the like. In some embodiments, a combination of the various EME is administered to the subject. In some embodiments port 632 can be connected to a helmet of face covering that is placed over the subject's head/face. It may include one or more screens that provide visual modality to help relax the subject. In some embodiments, this can be a flicker. In some embodiments, port 632 can be connected to earphones or directly provide music or other sounds to the patient. In some embodiments, the EME can be administered with a frequency corresponding to brain frequencies. While some embodiments described herein use port 632, it should be understood that the devices and modalities described in this paragraph can be wirelessly or electrically connected to a user interface or controller that may or may not be connected to the pump. In some embodiments, electrical impulses can be provided to the patient.

In some embodiments, MRI can be used to show the concentration of amyloid beta in a subject's brain. In some embodiments, Gd contrast agent is used prior to the MRI. Positron Emission Tomography (PET) with agents such as radioactive agents can also be used. In these embodiments, MRI or PET results, over time, can be used to show a decrease in the concentration of amyloid beta.

In some embodiments, to help a subject achieve the low brain activity state, tranquilizers, sedatives, or other chemicals that reduce brain activity can be used.

In some embodiments, the electrical or electromagnetic energy supplied to a subject has a frequency that corresponds to a subject's brain waves. In some embodiments, the frequency of the energy corresponds to the frequency of a subject's alpha, theta, and/or delta brain waves. In some embodiments, the sound energy supplied to a subject comprises music that has a calming effect of the patient and/or has a beat that corresponds to the frequency of a subject's brain waves. In some embodiments, the visual information provided to a subject includes images that have a calming effect on the subject. In some embodiments, the visual information is a series of flashes or pulses with a frequency that corresponds to the subject's brain wave frequency.

In some embodiments, a subject is positioned at a zero-gravity position. In some embodiments, this means that a body is in a neutral posture while the feet are elevated in alignment with the heart. In some embodiments, in this position, a subject's torso and thighs are at equal angles from the hip, the upper body is elevated, the knees are bent, and/or the legs are raised to about chest level. In this position, the spine is in a relatively stress-free position, the angle of the head is such that a subject's tongue is at a normal position, so breathing is easier, and/or with the legs raised slightly, blood flows more easily through a subject's body. For some subject's the zero-gravity position will decrease stress, lower blood pressure, and/or relax the brain. In some embodiments, the torso and/or thighs are each about 30 to 45 degrees away from the fully prone position. In some zero-gravity positions, the torso and thigh have an angle of 128+/−8 degrees, the hamstrings and calf are at an angle of 133+/−8 degrees, and/or the head is positioned at an angle of 24+/−5 degrees anteriorly from being aligned with the torso.

In some embodiments, the patient is positioned in the Trendelenburg position. In this position, a person is laid face up on a surface, and then the head in angled down. In some embodiments, the person is positioned at an angle of about 15° to about 20°. In some embodiments, the person is positioned at an angle of about 30° to about 40°.

In some embodiments, a subject is placed on a bed (flat, raised torso/head, raised knees, or a combination) for treatment by the products described herein and/or by the methods described herein. When positioned on the bed, the bed can be flat, inclined with the head raised, or inclined with the feet raised.

In some embodiments, the present invention provides a method for improving symptoms of Alzheimer's and other neurodegenerative diseases and disorders. Lowering the level of beta-amyloids and/or metabolic waste products in the neural space will results in improved symptoms. In some embodiments, the neurodegenerative diseases include different forms of dementia including Alzheimer's and Parkinson's and other diseases such as cognitive decline, MCI (mild cognitive impairment), MS (multiple sclerosis), and migraines. In some embodiments, these diseases/disorders include Alzheimer's, Parkinson's, Huntington's dementia, prion disease, and multiple sclerosis, brain injury, trigeminal neuralgia, tremors, bipolar disorder, attention deficit disorder, seizures, as well as memory loss, visuo-constructive capacities, and verbal fluency. In an embodiment, the disclosed methods counteract the problems associated with these conditions.

In an embodiment, a method for improving the symptoms of cognitive decline is provided. In some subjects, increased sagittal vertical axis measurements are correlated with cognitive decline. The sagittal vertical axis is the length of a horizontal line connecting the posterior superior sacral end plate to a vertical line from the centroid of the C7 vertebral body. The more the head and neck protrude in front of the pelvis when viewed from the side the greater the distance of this line from the C7 vertebral body. In some studies, the greater this distance, the more likely subjects are to show symptoms of cognitive decline. It is believed that this change in posture inhibits the natural pumping of CSF by the body as it is more difficult for a subject to flex their spine. By using the pumps described herein, CSF flux and flow can be increased to improve the symptoms of cognitive decline. In an embodiment, the use of the pumps described herein will lessen the amount of the sagittal vertical axis measurements. In an embodiment, the disclosed methods counteract the problems associated with this condition.

In an embodiment, an apparatus and method for improving posture is provided. The central nervous system and muscles are in constant communication to keep joints and limbs within a “safe” range of motion. One such safety mechanism is the presence of muscle spindle innervated by type 1A nerve fibers that surround muscles and communicate muscle stretch to the spinal cord and brain. If a stretch becomes excessive (e.g., arms swinging uncomfortably far behind your back), the spindle fibers activate specific motor neurons that cause the associated muscle(s) to rapidly contract, thereby bringing muscles/joints back to the normal range of motion. Another protective mechanism is provided by the Golgi tendon organs (GTOs), which sense the amount of load or tension on a muscle. If GTOs sense too high of a load (i.e., trying to move something too heavy), the GTOs will communicate to the spinal cord and inhibit the motor neuron's ability to contract the muscle, thereby preventing possible injury. GTOs are sensitive to changes in tension and rate of tension and because they are located in the musculotendinous junctions, they are responsible for sending information to the brain as soon as they sense an overload. Stretching is one example of how muscle tension signals a GTO response. When a low-force stretch is held for a period of time, the increase in muscle tension activates the GTO, which temporarily inhibits muscle spindle activity (thus reducing tension in the muscle) and allows for further stretching (reprogram the spindle fibers). In one embodiment, the change in flexure of the cervical spine will reprogram the muscles associated with posture and allow the subject to have improved posture as muscles that were accustomed to bad posture will more easily support a good posture position. In one embodiment, the spindle fibers are reprogrammed to allow for increased range of motion for the affected muscles.

In an embodiment, a method for decreasing neuroinflammation is provided. Neuroinflammation is an inflammatory response within the brain or spinal cord and occurs when the brain's innate immune system is triggered following an inflammatory challenge such as those posed by injury, infection, exposure to a toxin, various inflammatory agents or aging. Injuries, infections, toxins, various inflammatory agents, and aging are associated with decreased glymphatic system effectiveness. Some studies have shown a rising tau and beta amyloid level is associated with higher levels of neuroinflammation. In an embodiment, a method for reducing neuroinflammation is provided. Through the use of a CSF pump of the present disclosure, as the CSF system flux is increased and the glymphatic system performance is improved, the effects of injuries, infections, toxins, various inflammatory agents, and aging on the brain are decreased.

Some researchers believe that the increased amyloid beta production and plaque formation found in Alzheimer's and other neurodegenerative disease patients cause Alzheimer's and the other neurodegenerative diseases. Other researchers, however, believe that neuroinflammation of the brain tissue and/or brain trauma causes increased amyloid beta (and other waste product) production as a normal immune response. Chronic increased production leads to the formation of plaques and the onset of symptoms of Alzheimer's and other neurodegenerative diseases. As discussed herein, by using the CSF pumps describe herein, increased CSF flow/flux can improve symptoms of Alzheimer's and other neurodegenerative diseases by increasing the effectiveness of the glymphatic system. Today, some Alzheimer's drugs are antibodies that target a protein called amyloid-beta. Amyloid-beta protein accumulates in the brains of Alzheimer's disease patients, and it is thought to contribute to the cognitive decline in Alzheimer's disease. These antibodies help patients' immune systems to remove the amyloid-beta deposits from the brain. Other Alzheimer's drugs include cholinesterase inhibitors which prevent the breakdown of acetylcholine, a brain chemical believed to be important for memory and thinking. Other researchers, however, believe that the production of amyloid beta is caused by neuroinflammation as a normal anti-inflammatory immune response and, rather than a cause of Alzheimer's, is a symptom of Alzheimer's. These researchers have proposed that by administering these antibodies, the body actually produces more amyloid beta. In an embodiment, the use of antibodies in conjunction with improved glymphatic clearance will improve the symptoms of Alzheimer's and other neurodegenerative diseases. By increasing CSF flux and flow, glymphatic clearance will be improved and neuroinflammation will be decreased. Administering antibodies after reducing neuroinflammation will help rid the body of accumulated amyloid beta deposits and, with the reduced neuroinflammation, the natural systems of the body will produce less beta amyloid.

In an embodiment, a method for increasing the effectiveness of both the glymphatic and lymphatic systems is provided. As discussed herein, fluid and waste products from the brain/CNS flow into the CSF and then directly to the lymphatic system. There is also a flow path for fluid from the ISF system directly to the lymphatic system. Simultaneously, fluid and waste flows into the CSF from the ISF and then to the blood after being filtered by the arachnoid villi. When using the pumps described herein, CSF flux in increased. This increased CSF flux results in higher fluid/nutrient flow to the brain as the pressure of the CSF system will be increased. With more fluid flow to the brain, more fluid and waste products must flow out of the brain. Some of this exiting fluid and waste products flow directly to the lymphatic system and other fluid and waste flows through the arachnoid villa from CSF to the blood. By providing mild oscillating pressure to the human body, including the limbs, the lymphatic system is stimulated, and lymphatic fluid flow is increased. By providing this pressure to improve lymphatic system dynamics in conjunction with using a pump of the present disclosure to increase CSF flux, improvements in the symptoms of diseases are provided. In some embodiments, a lymphatic device that applies pressure to the upper body is used. In some embodiments, the CSF pump is used at the same time as the lymphatic device is used and, in some embodiments, they are used at different times.

As used herein, improve or improving symptoms means to lessen, slow, or eliminate the advancement of a disease, lessen or reduce some or all symptoms of a disease, cause the disease to go into remission, or cure a disease.

As humans age, the human body slows down and CSF flux and flow is decreased as compared to when the human was at a younger age. Like many systems, when a stress (reduced CSF flow and flux) is applied to a system, the weakest links will begin to fail. In humans, this may be expressed by having diseases or conditions that a human is predisposed to become more prevalent. For some people, this could be memory issues, problems sleeping, depression, or other abnormal brain activity. These conditions could also be accelerated by head or CNS trauma or injury. It is believed that stimulation of the brain can cause a release of growth or other hormones and/or enzymes. In some embodiments, improving the flow of CSF in the spinal column and/or brain will stimulate the brain. Some believe that CSF generation is a passive process—the body generates as much or as little as needed. If CSF flux and flow is increased, the body's filtering of CSF will increase causing an increase in CSF generation. Higher CSF circulation also results in brain stimulation and an increase in the release of growth or other hormones and/or enzymes. In some embodiments, as described herein, stimulation of the brain can be caused by providing electro/mechanical energy to a subject.

In an embodiment, an apparatus and method for improving the symptoms of Alzheimer's and other neurodegenerative diseases is disclosed. By increasing CSF flux and flow, which in turn increases the removal of brain and/or CNS waste products, symptoms of Alzheimer's and other neurodegenerative diseases are improved.

In an embodiment, a method of improving the symptoms of depression is provided. In an embodiment, a method for improving the sleep pattern is provided. In an embodiment, a method for improving memory is provided. For many conditions or diseases that are caused by the brain not working optimally, it is believed that increased CSF flux in the spinal column and/or in the brain will improve the working of the brain and then improve the symptoms of these conditions or diseases.

In an embodiment, an apparatus and method for increasing the speed and/or efficiency and/or effectiveness of delivery of drugs injected into the CSF is provided. For drugs injected or infused into the CSF system, the rate of drug transport is dependent upon the natural flow and flux of the CSF system. By using a pump of the disclosure to increase the flux of the CSF system, the rate of drug delivery is increased, and the efficiency of the drug may also be increased. In an embodiment, a method for increasing the speed and/or efficiency and/or effectiveness of intravenous chemotherapy drugs for conditions of the brain. By using a pump of the disclosure and increasing CSF flux, there is improved delivery of the intravenous drugs.

In an embodiment, an apparatus and method for improving the symptoms of chemo-brain is provided. Often chemotherapy patients will experience disorganized behavior or thinking, confusion, memory loss, and trouble concentrating, paying attention, learning, and making decisions. These signs and symptoms are referred to as chemo brain. By using a pump of the invention, the increase in CSF flux results in improved glymphatic clearance, removing the byproducts of the chemotherapy drugs from the brain.

In an embodiment, an apparatus and method for improving the health of patients who are bedridden is provided. People may be confined to a bed by sickness or old age. This can include people in a coma, people who are paralyzed, disabled, or otherwise immobilized. One way that the human body increases CSF flow and flux is through exercise or movement. When bedridden, the body cannot exercise and, with some conditions, cannot move. Some of the pump embodiments described herein are constructed in a manner that they are thin enough to be positioned on a regular or hospital bed mattress without the need for a specialized mattress. Some pump embodiment described herein would require a modified mattress, perhaps a mattress with an opening to allow for a pump to be positioned within the opening. To increase the CSF flow of a bedridden patient, a pump of this invention can be positioned near the spine, in some embodiments near the lumbar region, and activated to cause a change in flexure of the spinal column. This change in flexure of the spinal column will result in increased CSF flow and improved glymphatic clearance. In instances where a brain injury or neurodegenerative disease causes a patient to be bedridden, the improve glymphatic clearance may also reduce the symptoms of the brain injury and/or neurodegenerative disease.

In some embodiments, a control system will be associated with the pump or pumps. In some embodiments when using a single pump, the spinal column will be flexed 1 to 5 mm, or from 2 to 4 mm, or about 3 mm from the at rest (shallow breathing) position. In some embodiments when using a lumbar or thoracic pump and a cervical pump, the lumbar or thoracic region of the spinal column will be flexed 1 to 5 mm, or from 2 to 4 mm, or about 3 mm from the at rest (shallow breathing) position and the cervical region of the spinal column will be flexed by providing flexion and extension to a subject's head in the range of 10 to 40 degrees, or 15 to 35 degrees, or 20 to 30 degrees away from the neutral position. In some embodiments, the amount of flexion may be more or less or equal to the amount of extension. In embodiments where lateral flexion is provided, flexing in the amount of 10 to 45 degrees, or 15 to 40 degrees, or 20 to 30 degrees from the neutral position may be used. In some embodiments, the flexing of the spinal column may start at a low level and be increased over the course of a treatment. A subject may receive from 1 to 7 or more treatments in a week. Each treatment will be 15, 20, 25, 30, 35, 40, or 45 minutes or more.

In some embodiments, a pump is used to create a change in pressure and/or a pressure gradient and/or a pressure wave in the CSF system without removing any CSF from the CSF system. In these embodiments, it may be safer for a patient to receive the benefits of increased CSF flux and flow without the surgery needed to access and remove CSF from the CSF system, even if the removed CSF is ultimately returned to the CSF system.

In one non-limiting example, a subject is placed in a chair as described herein and the subject is rotated to a zero-gravity position. The chair can include a cervical flexure device and a lumbar flexure device. The subject is positioned on the chair and the lumbar flexure device is positioned adjacent to L2 and/or L3. A control system that controls the two flexure devices raises the lumbar flexure device, while the cervical flexure device positions the subject's head in the neutral position, just until the pad of the lumbar flexure device touches the cloths/skin of the subject's posterior body. This is called the home position. In some embodiments of this non-limiting example, a technician will raise the lumbar flexure device until the pressure on the lumbar just exceeds comfortable but not so far as to cause pain for the patient. This distance is the span that the lumbar flexure device will travel during treatment. In some embodiments of this non-limiting example, the technician or the control system will set the span distance between 20 and 30 mm or between 20 and 25 mm. The control system will have a limit of about 40° extension and 40° flexion for the cervical flexure device or about 30° for extension and 30° for flexion. The technician will then activate the control system. The first movement will be the cervical flexure device moving to a position of flexion. The control system will simultaneously cause the lumbar flexure device to flex the spinal column a distance equal to the span while the cervical flexure device moves from flexion to extension. Then the control system will simultaneously cause the lumber flexure device to return to the home position while the cervical flexure device moves from extension to flexion. These two movements are called one cycle. In some embodiments of this non-limiting example, the complete cycle will take between 10 and 15 seconds, with the upward movement (away from the home position) of the lumbar flexure device will take about 35% to 45% of the cycle time and the downward movement (back to home position) will take 55% to 65% of the cycle time. In some embodiments of this non-limiting example, the treatment time will be between 30 and 45 minutes. In some embodiments of this non-limiting example, the subject will receive five treatments per week. In some embodiments of this non-limiting example, the span of the lumber flexure device will be set to 20 mm at the start of the treatment and will gradually be increased to 30 mm by the end of the treatment.

Although specific embodiments have been illustrated and described herein, those of ordinary skill in the art will appreciate that an arrangement calculated to achieve the same results can be substituted for the specific embodiments shown. For example, where the disclosure my show a system or a method with one example of a distal protection device, any distal protection device can be used including those disclosed herein. This disclosure is intended to cover adaptations or variations of one or more embodiments of the present disclosure. It is to be understood that the above description has been made in an illustrative fashion, and not a restrictive one. Combination of the above embodiments, and other embodiments not specifically described herein will be apparent to those of skill in the art upon reviewing the above description. The scope of the one or more embodiments of the present disclosure includes other applications in which the above structures and processes are used. Therefore, the scope of one or more embodiments of the present disclosure should be determined with reference to the appended claims, along with the full range of equivalents to which such claims are entitled.

In the foregoing Detailed Description, some features are grouped together in a single embodiment for the purpose of streamlining the disclosure. This method of disclosure is not to be interpreted as reflecting an intention that the disclosed embodiments of the present disclosure have to use more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. An apparatus comprising:
a pump configured in a pumping state to change dynamics of a cerebrospinal fluid (CSF) system of a subject as compared to the dynamics of the CSF in a non-pumping state.

2. The apparatus of claim 1, further comprising:
a backrest configured to support a back of the subject;
an aperture in the backrest; and
a moveable pad configured to extend through the aperture to pump the CSF system.

3. The apparatus of claim 2, wherein the moveable pad is configured to move in a direction perpendicular to a plane of the backrest.

4. The apparatus of claim 1, wherein the pump is configured to flex a spinal column of the subject from 1 to 5 cm.

5. The apparatus of claim 1, wherein the pump is configured to provide flexion and extension from about 10 to about 40 degrees from a neutral position of a spine of the subject.

6. The apparatus of claim 1, wherein the pump is characterized as a first pump, further comprising:
a backrest configured to support a back of the subject;
an opening in the backrest, the first pump positioned in the opening;
a headrest configured to support a head of the subject; and
a second pump configured to move the headrest in relation to the backrest.

7. The apparatus of claim 6, wherein when the first pump is extended the second pump is in extension, and when the first pump is retracted the second pump is in flexion.

8. The apparatus of claim 6, wherein the headrest is configured to rotate with respect to the backrest.

9. The apparatus of claim 6, wherein a rotation axis of the second pump is oriented orthogonal to an actuation axis of the first pump.

10. An apparatus comprising:
a pump configured in a pumping state to oscillate between and first position and second position to change dynamics of a cerebrospinal fluid (CSF) system of a subject as compared to the dynamics of the CSF in a non-pumping state.

11. The apparatus of claim 10, further comprising:
a pressure pad configured to provide pressure to a spinal column of the subject;
a first support configured to support the subject; and
a second support configured to support the subject, wherein in a first orientation the pressure pad is in a retracted position and the second support is oriented at a first angle to the first support; and in a second orientation the pressure pad is in an intermediate position and the second support is oriented at a second angle to the first support, the second angle being greater than the first angle.

12. The apparatus of claim 10, wherein the pump is configured to move in an anterior direction for a duration of 3 to 8 seconds and a posterior direction for a duration of 3 to 8 seconds.

13. The apparatus of claim 10, wherein the pump is configured for complete cycling between 10 and 15 seconds between a first position and a second position.

14. The apparatus of claim 10, wherein the pump is configured to move a spinal column of the subject from 1 to 5 cm in an anterior direction.

15. The apparatus of claim 10, wherein the pump is configured to provide flexion and extension to a head of the subject in range of 10 to 40 degrees away from a neutral position of the spine of a subject.

16. The apparatus of claim 10, wherein a home position of the pump is configured to put the subject in home position, and a cycle time will take between 10 and 15 seconds, with an upward movement away from the home position of a lumbar flexure device will take about 50% of the cycle time and a downward movement back to the home position will take 50% of the cycle time.

17. The apparatus of claim 10, further comprising:
a shaft; and
a pressure pad coupled to the shaft, wherein the pump is configured to move the pressure pad back and forth in an axial direction.

18. An apparatus comprising:
a pump configured to oscillate a headrest configured to support a head of a subject, the headrest is configured to rotate between extension and flexion in a pumping state to change dynamics of a cerebrospinal fluid (CSF) system of a subject as compared to the dynamics of the CSF in a non-pumping state.

19. The apparatus of claim 18, further comprising:
a backrest configured to support a back of the subject;
an aperture in the backrest; and
a moveable pad configured to extend through the aperture to pump the CSF system.

20. The apparatus of claim 19, wherein the moveable pad is configured to move in a direction perpendicular to a plane of the backrest.

21. The apparatus of claim 20, wherein the moveable pad is configured to flex a spinal column of the subject from 1 to 5 cm.

22. The apparatus of claim 18, wherein the pump is configured to provide flexion and extension from about 10 to about 40 degrees from a neutral position of a spine of the subject.

23. The apparatus of claim 18, wherein an extension of the headrest away from a home position will take about 35% to 45% of a cycle time of an oscillation and a flexion of the headrest back to the home position will take 55% to 65% of the cycle time of the oscillation.

24. The apparatus of claim 18, wherein a cycle time of an oscillation will take between 10 and 15 seconds.

25. An apparatus comprising:
a frame;
a support coupled to the frame, the support configured to support a body of the subject;
an aperture in the support;
a cerebrospinal fluid pump attached to the frame, the cerebrospinal fluid pump configured to move with respect to the support along an actuation axis between a retracted position and an extended position.

26. The apparatus of claim 25, wherein the support is characterized as a first support, further comprising:
a second support configured to support a head of the subject and to rotate with respect to the first support about a rotation axis.

27. The apparatus of claim 26, the rotation axis is oriented orthogonal to the actuation axis.

28. The apparatus of claim 26, comprising:
a first orientation wherein the cerebrospinal fluid pump is in the retracted position and the second support is oriented at a first angle to the first support; and
a second orientation wherein the cerebrospinal fluid pump is in an intermediate position and the second support is oriented at a second angle to the first support, the second angle being greater than the first angle.

29. The apparatus of claim 25, wherein the cerebrospinal fluid pump is configured to extend through the aperture.

30. The apparatus of claim 25, wherein the cerebrospinal fluid pump is configured to oscillate between an upward movement away from a home position that will take about 35% to 45% of a cycle time and a downward movement back to the home position that will take 55% to 65% of the cycle time.
